(19) United States
(12) Patent Application Publication
(10) Pub. No.: US 2025/0235259 A1
(43) Pub. Date: Jul. 24, 2025

(54) SPHINCTEROTOMES WITH DAMAGE RESISTANT CUTTING EDGE COVERS

(71) Applicant: Cook Medical Technologies LLC, Bloomington, IN (US)

(72) Inventors: Tyler McLawhorn, Winston-Salem, NC (US); Michael Entz, Winston-Salem, NC (US); Rachel Welscott, Jacksonville, FL (US); Hunter Pelham, Winston-Salem, NC (US)

(73) Assignee: Cook Medical Technologies LLC, Bloomington, IN (US)

(21) Appl. No.: 19/024,517

(22) Filed: Jan. 16, 2025

Related U.S. Application Data
(60) Provisional application No. 63/622,265, filed on Jan. 18, 2024.

(51) Int. Cl. A61B 18/14; A61B 18/00; A61B 18/12

(52) U.S. Cl. CPC A61B 18/1492; A61B 2018/00083; A61B 2018/00136; A61B 2018/00184; A61B 2018/00482; A61B 2018/00601; A61B 2018/1253; A61B 2018/126; A61B 2018/144; A61B 2018/1475

(57) ABSTRACT

A sphincterotome includes a variable thickness cover that covers an insulated portion of a cutting edge. The variable thickness cover includes a distal taper distally extending from a first outer diameter to a second outer diameter, where the first outer diameter is larger than the second outer diameter.

### CROSS-REFERENCE TO RELATED APPLICATIONS

This application claims the benefit of U.S. Provisional Application No. 63/622,265, filed Jan. 18, 2024. The contents of U.S. Provisional Application No. 63/622,265 are herein incorporated by reference in their entirety.

### TECHNICAL FIELD

The present invention relates generally to medical devices, and more particularly to sphincterotomes.

### BACKGROUND

A sphincterotome is a medical device that is used to perform a sphincterotomy, which involves cutting a sphincter muscle, such as the sphincter of Oddi. The sphincter muscle may need to be cut to relieve its constrictive nature and allow one or more medical devices through the muscle. For example, problems occurring in the biliary tree, such as the formation of bile duct stones or papillary stenosis, may be treated using medical devices that are delivered into the biliary tree. In order to access the biliary tree, the medical devices may pass through the sphincter of Oddi. To facilitate passage of the medical devices through the sphincter of Oddi, the sphincter muscle may be cut using a sphincterotome.

### BRIEF SUMMARY

The present describes various embodiments of a sphincterotome, electrosurgical systems, and electrosurgical medical devices and related methods that have a damage-resistant cover covering an insulated portion of a cutting edge.

In one embodiment, a sphincterotome includes: an elongate tubular member longitudinally extending from a proximal portion to a distal portion, the elongate tubular member comprising: a body longitudinally extending from the proximal portion to the distal portion; and a cutting wire lumen longitudinally extending in the body; a cutting wire longitudinally extending and movably disposed in the cutting wire lumen, the cutting wire comprising a cutting edge disposed outside of the elongate tubular member, the cutting edge comprising an exposed portion and an insulated portion; a variable thickness cover that covers the insulated portion, the variable thickness cover comprising a distal taper distally extending from a first outer diameter to a second outer diameter, the first outer diameter larger than the second outer diameter.

In another embodiment, a sphincterotome includes: an elongate tubular member longitudinally extending from a proximal portion to a distal portion, the elongate tubular member comprising: a body longitudinally extending from the proximal portion to the distal portion; and a cutting wire lumen longitudinally extending in the body; a cutting wire longitudinally extending and movably disposed in the cutting wire lumen, the cutting wire comprising a cutting edge disposed outside of the elongate tubular member, the cutting edge comprising an exposed portion and an insulated portion; a multi-layer cover that covers the insulated portion, the multi-layer cover comprising a first layer disposed over the insulated portion, and a second layer disposed over the first layer.

Other embodiments are possible, and each of the embodiments can be used alone or together in combination. Accordingly, various embodiments are described below with reference to the accompanying drawings.

### BRIEF DESCRIPTION OF THE DRAWINGS

FIG. 1 shows a cross-sectional side view of an electrosurgical system including a sphincterotome electrically coupled to a power source.

FIG. 2 shows a perspective view of a distal portion of the sphincterotome of FIG. 1 in a curled position.

FIG. 3 shows cross-sectional side view of the distal portion of the sphincterotome of FIGS. 1 and 2, illustrating a first example configuration of a cover of a cutting edge, where the cover of the first configuration is a variable-thickness cover having a distal taper.

FIG. 4 shows a first example configuration of the variable-thickness cover of FIG. 3.

FIG. 5 shows a second example configuration of the variable-thickness cover of FIG. 3.

FIG. 6 shows a third example configuration of the variable-thickness cover of FIG. 3.

FIG. 7 shows a fourth example configuration of the variable-thickness cover of FIG. 3.

FIG. 8 shows a fifth example configuration of the variable-thickness cover of FIG. 3.

FIG. 9 shows another configuration of a variable thickness cutting edge cover, where the distal taper has a continuously decreasing outer diameter.

FIG. 10 shows a cross-sectional side view of the distal portion of the sphincterotome of FIGS. 1 and 2, illustrating a second example configuration of the cover of the cutting edge, where the cover of the second configuration is a multi-layer cover.

### DETAILED DESCRIPTION

The present description describes various embodiments of a sphincterotome, electrosurgical systems, electrosurgical medical devices, and related methods having a damage-resistant cutting edge cover. The damage-resistant cutting edge covers described herein may decrease the likelihood that the cutting-edge cover will catch on another portion of the sphincterotome or on an endoscope or its elevator, compared to other cutting edge protectors, which in turn may prevent the protector from becoming damaged to the extent that it undesirably exposes a portion of the cutting edge that it was covering. As a result, undesired sparking that may otherwise occur from such exposure may be avoided, which in turn may minimize undesired harm to the patient during a sphincterotomy.

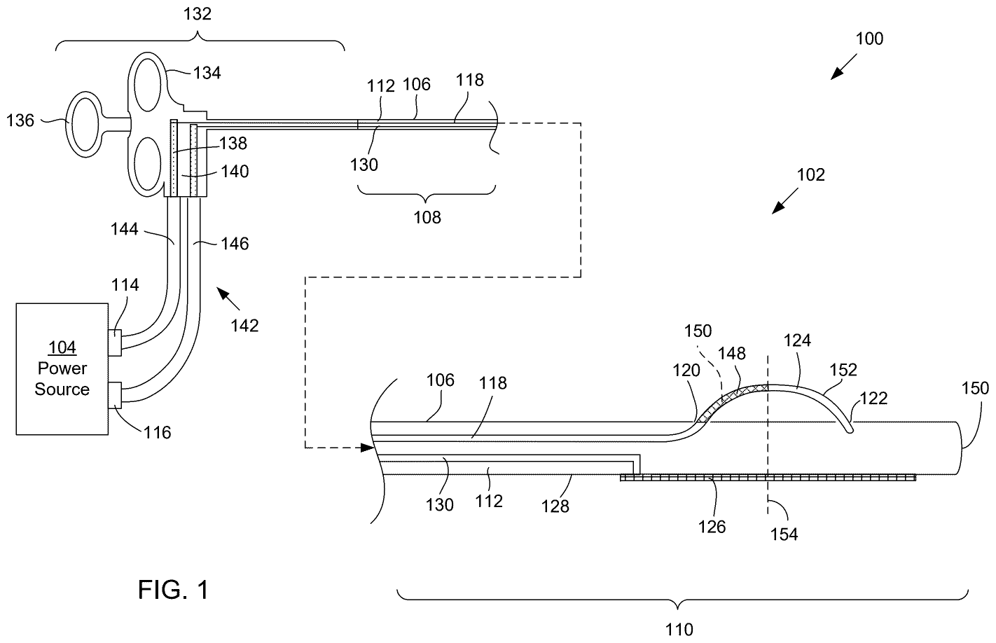

FIG. 1 shows a partial cross-sectional side view of an example electrosurgical system 100 that includes a sphincterotome 102 coupled to a power source 104. Other embodiments of the present description may include only the sphincterotome 102 without the power source 104. Additionally, in some embodiments such as in FIG. 1, the sphincterotome 102 has a bipolar configuration (in which case the sphincterotome 102 is referred to herein as a bipolar sphincterotome). In other embodiments, the sphincterotome 102 has a monopolar configuration (in which case the sphincterotome 102 is referred to herein as a monopolar sphincterotome). Further details of bipolar and monopolar configurations for the sphincterotome 102 are described below.

The sphincterotome 102 may include an elongate tubular member 106 (e.g., a catheter) that longitudinally extends from a proximal portion 108 to a distal portion 110. The elongate tubular member 106 may include a body 112 extending from the proximal portion 108 to the distal portion 110. The body 112 may be made of various suitable materials, including non-conductive materials, such as polytetrafluoroethylene (PTFE), perfluoroalkoxy (PFA), polyethylene, nylon, or fluorinated ethylene, as non-limiting examples.

The bipolar sphincterotome 102 may further include an active path and a return path. In general, the active path and the return path are each conductive elements, or each a network of elements, that are configured to deliver electrical current between the power source 104 and a treatment site (e.g., a sphincter muscle) within a patient. As shown in FIG. 1, the power source 104 (e.g, an electrosurgical unit (ESU) or a radio frequency (RF) generator) may be configured to output electrical current via a pair of ports, including an active port 114 and a return port 116. The active path may be configured to electrically couple to the active port 114, and the return path may be configured to electrically couple to the return port 116.

In addition, at a treatment site within a patient, a distal portion of the active path is configured to contact a portion of tissue at the treatment site. The configuration of the return path may depend on whether the sphincterotome 102 has a bipolar or a monopolar configuration. For a bipolar configuration, a distal portion of the return path is configured to contact another portion of the tissue. For a monopolar configuration, only the active path is configured to contact tissue. The return path extends outside of the patient, from a pad attached to the patient's skin, such as on the patient's leg to the return port 116 of the power source 104. As mentioned, the sphincterotome 102 described with reference to the figures has a bipolar configuration. However, the damage-resistant cutting edge covers described in further detail below can be similarly implemented in monopolar sphincterotomes.

In further detail, for a bipolar configuration such as in FIG. 1, when the distal portions of the active and return paths are contacting the tissue, the power source, the active and return paths, and the tissue at the treatment site form an electrical circuit through which electrical current generated by the power source 104 may flow. The electrical current flowing through the tissue may produce a certain effect on the tissue, such as cutting, ablation, or coagulation, as non-limiting examples. In addition, for an electrosurgical procedure such as a sphincterotomy, the electrical current may be an alternating current (AC), such as a radio frequency (RF) current. Accordingly, when the electrical circuit is formed, depending on its polarity, electrical current generated by the power source 104 may flow from the active port 114 of the power source 104, through the active path of the bipolar sphincterotome 102, through the tissue at the treatment site, and then through the return path back to the return port 116; or may flow from the return port 116, through the return path, through the tissue at the treatment site, and then through the active path back to the active port 114.

Additionally, the active path of the sphincterotome 102 may include a conductive cutting wire 118 (also called an active wire) that longitudinally extends from the proximal portion 108 to the distal portion 110. In various embodiments, such as shown in FIG. 1, the cutting wire 118 may longitudinally extend within the body 112 except at the distal portion 110, where the cutting wire 118 exits at an exit port 120 from within the body 112 to outside the body 112, and distally extends outside the body 112 from the exit port 120 to an anchor point 122, where a distal end of the cutting wire 118 is secured or affixed to the body 112. The portion of the active wire 118 that is outside of the body 112 may be referred to as a cutting edge 124, and is an electrode part of the active wire 118 that is configured to contact tissue at the treatment site. For example, during a sphincterotomy, the cutting edge 124 is a part of the active wire 118 that is configured to contact a sphincter muscle and cut the sphincter muscle when electrical current is delivered to it. In various embodiments, the cutting edge 124 may have a diameter in a range of about 0.010 inches to about 0.024 inches, although other diameters may be possible in other embodiments. Additionally, in various embodiments, a remaining portion of the active wire 118 other than the cutting edge 124 may have the same diameter or a different diameter than the cutting edge 124.

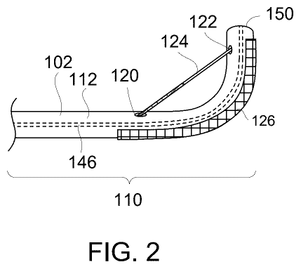

In addition, the cutting wire 118 may be longitudinally movable within the body 112, such as by being disposed within an active wire lumen (not shown in FIG. 1) that longitudinally extends from the proximal portion 108 to the distal portion 110. By being longitudinally movable within the body 112, the cutting wire 118 may be configured to move the distal portion 110 of the elongate tubular member 106 between curled (or bowed) and uncurled positions. FIG. 1 shows the distal portion 110 in an uncurled position. FIG. 2 shows the distal portion 110 in a curled position. For example, when the cutting wire 118 is proximally pulled relative to the elongate tubular member 106, the distal end of the cutting wire 118 may exert a force on the elongate tubular member 106 at the anchor point 122 that causes the distal portion 110 to curl in a curling direction, as shown in FIG. 2. When the distal portion 110 is in a curled position due to the active wire 118 being proximally pulled, the cutting edge 124 may be relatively taut and in a cutting position. An operator operating the sphincterotome 102 to perform a sphincterotomy may operate the sphincterotome 102 to proximally pull the cutting wire 118 in order to configure the distal portion 110 in the curled position and the cutting edge 124 to be taut in the cutting position in order to cut the tissue.

As described, the sphincterotome 102 shown in FIGS. 1 and 2 has a bipolar configuration, in that its return path is integrated with, or a part of, the elongate tubular member 106. For example, the return path of the bipolar sphincterotome 102 may include a return electrode 126 at the distal portion 110. In general, the return electrode 126 is a conductive component of the return path that is exposed from within the elongate tubular member 106 and is configured to contact tissue at the treatment site. Additionally, in various embodiments, such as shown in FIG. 1, the return electrode 126 may cover, be disposed over or about, and/or affixed or adhered to an outer surface 128 of the body 112 of the elongate tubular member 106. The return electrode 126 may be made of any various conductive materials, such as conductive ink, paste, foil, wires, or sheet metal, as non-limiting examples. Also, although not expressly shown in the figures, the bipolar sphincterotome may include one or more intermediate layers, such as an epoxy layer, that adheres the conductive material of the return electrode 126 to the outer surface 128 of the body 112. In this context, the return electrode 126 covering, being disposed over or about, and/or affixed or adhered to the outer surface 128, does not necessarily require that the conductive material of the return electrode 126 is in direct contact with the outer surface 128 of the body 112. Additionally, in various embodiments, the return electrode 126 may extend a longitudinal length over which the cutting edge 124 also longitudinally extends, from the exit port 120 to the anchor point 122. Also, for at least some of these embodiments, the return electrode 126 may further longitudinally extend at least one of: proximally past the exit port 120 of the elongate tubular member 106 or distally past the anchor point 122. FIGS. 1 and 2 show the return electrode 126 both proximally extending past the exit port 120 and distally extending past the anchor point 122.

Additionally, for the bipolar sphincterotome 102 of FIGS. 1 and 2, the return path may include a return wire or other elongate conductive element 130 that longitudinally extends from the proximal portion 108 to the distal portion 110 and that connects to the return electrode 126. In general, the return wire 130 longitudinally extends alongside the cutting wire 118 from the proximal portion 108 to the distal portion 110. In various embodiments, such as shown in FIG. 1, the return wire 130 may longitudinally extend within the body 112, such as by extending within a return lumen (not separately shown from the return wire 130 in FIGS. 1 and 2) of the elongate tubular member 106. In other embodiments, at least a portion of the return wire 130 extends outside, such as alongside, the body 112. Various ways of connecting the return wire 130 to the return electrode 126 and/or having the return path longitudinally extend from the proximal portion 108 to the distal portion 110 may be possible.

In other embodiments, the sphincterotome 102 may have a monopolar configuration, as previously described. For such configurations, the return path, including the return electrode 126 and the return wire 130, may be omitted or not part of the sphincterotome 102, such that the return path is not integrated with the sphincterotome 102. Instead, the return path may extend outside of the patient, from a return pad attached to the patient's skin to the return port 116, as previously described.

Additionally, the sphincterotome 102 may include a handle assembly 132 operably coupled to the elongate tubular member 106 and the cutting wire 118. In various embodiments, the handle assembly 132 may include a plurality of portions that are movable relative to each other to control longitudinal movement of the active wire 118 relative to the elongate tubular member 106. For example, in the example configuration shown in FIG. 1, the handle assembly 132 may include a first portion 134 in the form of finger rings and a second portion 136 in the form of a thumb ring through which an operator may insert his/her fingers and thumb, respectively, when grasping the handle assembly 132. The finger rings 134 may be operatively coupled to the cutting wire 118, such that movement of the finger rings 136 relative to the rest of the handle assembly 132 may cause the cutting wire 118 to longitudinally move relative to the elongate tubular member 106. Correspondingly, upon grasping the handle assembly 132, the operator may open and close his/her hand to move the finger rings 134 farther away and closer to the thumb ring 136, respectively. Moving the finger rings 134 toward the thumb ring 136 may proximally pull the cutting wire 118, which in turn may move the distal portion 110 into the curled position, such as shown in FIG. 2. Additionally, moving the finger rings 134 away from the thumb ring 136 may move the distal portion 110 into the uncurled position, such as shown in FIG. 1. The handle assembly 132 shown in FIG. 1 is merely exemplary, and various other configurations of a handle assembly to facilitate longitudinal movement of the cutting wire 118 relative to the elongate tubular member 106 may be possible.

Also, in various embodiments such as shown in FIG. 1, the handle assembly 132 may include components of the active path. For example, a proximal portion of the cutting wire 118 may extend in the handle assembly 132. Additionally, as shown in FIG. 1, the handle assembly 132 may include a conductive active element (or an assembly of active elements in various embodiments) 138 that connects to the cutting wire 118, and that is used to electrically couple the cutting wire 118 to the active port 114 of the power source 104. For bipolar configurations, such as shown in FIG. 1, the handle assembly 132 may similarly include a conductive return element (or an assembly of return elements in various embodiments) 140 that connects to the return wire 130, and that is used to electrically couple the return wire 130 to the return port 116 of the power source 104.

Additionally, in various embodiments, the electrosurgical system 100 may include electrical cabling 142 configured to electrically couple the power source 104 to the bipolar sphincterotome 102. The electrical cabling 142 may include an active cable or wire 144 configured to connect the active port 114 to the active path of the handle assembly 132 and/or sphincterotome 102. For example, the active cable 144 may connect to the active element 138 of the handle assembly 132. Similarly, for bipolar configurations such as in FIG. 1, the electrical cabling 142 may include a return cable or wire 146 configure to connect the return port 116 to the return path of the handle assembly 132 and/or sphincterotome 102. For example, the return cable 146 may connect to the return element 140 of the handle assembly 132. In various embodiments, the electrical cabling 140 may be considered a component of the bipolar sphincterotome 102. In other embodiments, the electrical cabling may be considered a component of the electrosurgical system 100 that is separate from the bipolar sphincterotome 102. In various embodiments, the active cable 144 and/or the return cable 146 may be fixedly connected or attached to the handle assembly 132. In other embodiments, the active cable 144 and/or the return cable 146 may be removably connected or attached to the handle assembly 132.

In addition, as shown in FIGS. 1 and 2, the sphincterotome 102 may include a cover 148 that covers and/or is disposed over a portion of the cutting edge 124. As used herein the portion of the cutting edge 124 that is covered by the cover 148 is referred to as an insulated portion 150, and the portion of the cutting edge 124 that is not covered by the cover 148 is referred to an exposed portion 152.

The cover 148 may prevent the portion of the cutting edge 124 it is covering from contacting tissue and delivering electrical current to the tissue. The cover 148 may serve as and also be referred to as a protector, in that it insulates and/or protects tissue from coming into contact with and being cut by the portion of the cutting edge 124 being covered by the cover 148. Accordingly, by having a portion covered by the cover 148, the entirety of the cutting edge 124 (e.g., the portion of the cutting wire 118 disposed outside of the elongate tubular member 106) is not configured to contact and cut tissue. The cover 148 may reduce the surface area of the cutting edge 124 configured or able to contact tissue, compared to if no cover was present. This, in turn, may improve the cutting ability of the cutting edge 124, and the overall ability of the sphincterotome 102 to perform a sphincterotomy. In particular, the cover 148 may improve the enhance performance of the sphincterotome 102 by: concentrating the current density to a smaller portion of the cutting edge 124 able to contact tissue, by preventing the proximal portion of the cutting edge 124 from cutting tissue that is not desired to be cut, and/or by preventing undesirable sparking.

In one configuration, the cover 148 is a single tubular member or sleeve with a single outer diameter. Such a configuration may be susceptible to becoming damaged such that it exposes at least a part of the cutting edge 124 it was covering before becoming damaged. The damage may be caused by contact, such as rubbing, between the cover 148 and a distal part of an endoscope (not shown in the figure) delivering the distal portion 110 of the sphincterotome 102 to the treatment site. For example, the endoscope may have a side exit port through which the distal portion 110 exits the endoscope, and/or may have an elevator that adjusts the positioning of the distal portion 110 to facilitate cannulation. Through movement of the distal portion 110 through the endoscope exit port and/or engagement with the elevator, the cover 148 may experience damage, exposing a part of the cutting edge 124 that was being covered. Such damage may lead to the above-mentioned problems that the cover 148 was otherwise configured to prevent, and in turn reduce the cutting ability of the sphincterotome 102 and/or increase the possibility of harm to the patient.

The following describes various configurations of the cover 148 that provide enhanced damage resistance compared a cover having a single tubular member and/or a single thickness. The enhanced damage resistance minimizes or decreases the likelihood that the portion of the cutting edge 124 to be covered remains covered during use of the sphincterotome, in turn maintaining the optimized cutting ability of the sphincterotome 102.

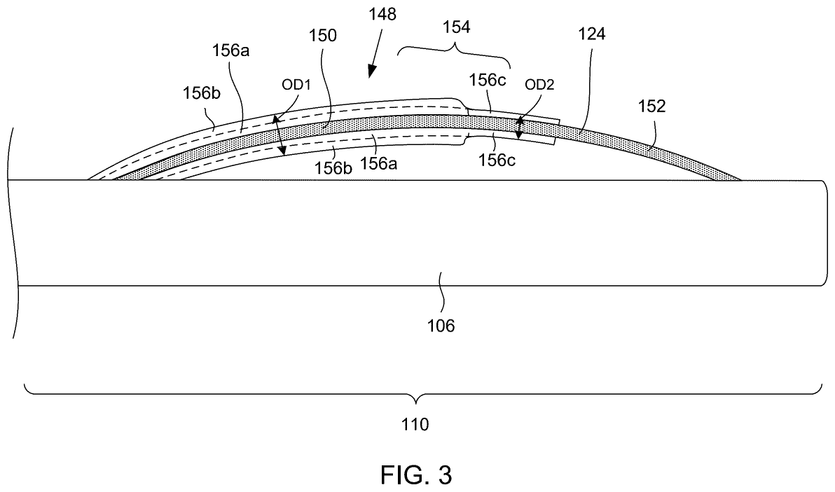

FIG. 3 shows a cross-sectional side view of the cutting edge 124 and an example configuration of the damage-resistant cover 148 at the distal portion 110. For clarity and simplicity, features internal to the elongate tubular member 106 are not shown. In the example configuration of FIG. 3, the cover 148 may be a variable thickness cover in that it has multiple outer diameters, including a first outer diameter indicated by double arrow OD1 and a second outer diameter indicated by double arrow OD2. The first outer diameter OD1 is larger or greater than the second outer diameter OD2. In this way, a portion of the cover 148 having the first outer diameter OD1 is thicker than the portion of the cover 148 having the second outer diameter OD2. For at least some embodiments, the first outer diameter OD1 is in a range of about 0.014 inches to about 0.02 inches, and the second outer diameter is in a range of about 0.011 inches to about 0.017 inches.

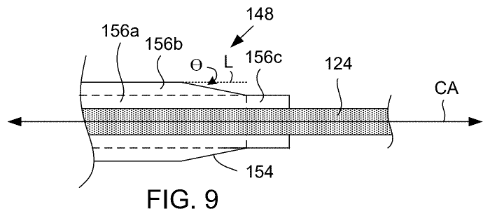

In addition, the variable thickness cover 148 may have or otherwise define a distal taper 154 over at least a portion of the cover 148 having two different outer diameters. For example, the cover 148 may have or define a distal taper over a portion that distally extends from the first outer diameter OD1 to the second OD2. In some embodiments, the distal taper 154 of the cover 148 has a staircase configuration, in that it “steps down” or sharply decreases from the first outer diameter OD1 to the second outer diameter OD2. In other embodiments, the distal taper 154 has a gradually or continuously decreasing outer diameter from the first outer diameter OD1 to the second outer diameter OD2. For example, as shown in FIG. 9, the distal taper 154 may be defined by an angle ⊖ relative to a line L parallel to a central axis CA of the cutting edge 124. The distal taper 154 may be considered gradual in that the angle ⊖ is less than or equal to 45 degrees. Various ways of configuring a variable thickness cover to have a distal taper that distally extends from a larger outer diameter to a smaller outer diameter may be possible.

In addition, in some embodiments, the cover 148 may include three portions 156, including a first portion 156a, a second portion 156b, and a third portion 156c. The first portion 156a may include or be made of a first material, and may be disposed directly over the insulated portion 150 of the cutting edge 124. The second portion 156b may include or be made of a second material, and may be disposed over the first portion 156a. The second portion 156b may be considered to overlap the first portion 156a. The third portion 156c may include or be made of a third material, and be disposed directly over the insulated portion 150 of the cutting edge 124, and be disposed distal the first portion 156. As shown in FIG. 3, the first and second portions 156a, 156b may define the first outer diameter OD1, and the third portion 156c may define the second outer diameter OD2. Also, in any of various embodiments, the first outer diameter OD1 may be variable (i.e., not constant) over the longitudinal length that the second portion 156b overlaps the first portion 156a, and/or the second outer diameter OD2 may be variable over the longitudinal length of the third portion 156c, including the distal taper 154 of the cover 148 that longitudinally transitions from the first and second portions 156a, 156b to the third portion 156c.

Additionally, in various embodiments, the first, second, and third materials of the first, second, and third portions 156a, 156b, 156c may be the same as or different from each other in any of various combinations. For example, in some embodiments, the first and second materials are the same. In other embodiments, the first and second materials are different. In addition or alternatively, in some embodiments, the third material is different than both the first and second materials. In other embodiments, the third material is the same as at least one of the first material or the second material. For example, in particular embodiments, the third material is the same as the second material.

Also, in any of various embodiments, the first material, the second material, and the third material are each, or each include, one of: parylene, polyimide, or polytetrafluoroethylene (PTFE). In addition or alternatively, in any of various embodiments, at least one of the first, second, or third materials is or includes a heat shrink material. Or, otherwise stated, in any of various embodiments, at least one of the first, second, or third portions 156a, 156b, 156c is a heat shrink or a heat shrink structure, such as a heat shrink tube. For example, in some embodiments, at least the first portion 156a and the second portion 156b are each a heat shrink. In other embodiments, at least the second portion 156b and the third portion 156c are each a heat shrink. Also, in general as used herein, a heat shrink structure is made of a heat shrink material and has a dimension, such as an outer diameter, that shrinks from an initial dimension value to a smaller or shrunken value in response to heat. For a given portion 156 of the cover 148 that is a heat shrink, the given portion 156 is in a heat shrunk state around the cutting edge 124 and applying an inward mechanical bias to remain in a fixed position over the cutting edge 124. Non-limiting examples of heat shrink materials may include polyolefine, fluoropolymer (such as PTFE), polyvinyl chloride (PVC), or neoprene, although other suitable heat shrink materials for one or more portions of the cover 148 may be possible. Additionally, such a heat shrink structure may be in contrast to a given portion 156 that is formed through a spraying or other depositing processes where a chemical bond is formed that affixes the given portion 156 to another portion 156.

In addition, in some embodiments, the third portion 156c may have a longitudinal length in a range of 0.5 millimeters (mm) to 15 mm. In other embodiments, the third portion 156c may have a longitudinal length in a range of 0.5 mm to 2.5 mm. In other embodiments, the third portion 156c may have a longitudinal length in a range of 2 mm to 10 mm. In addition or alternatively, in some embodiments, a ratio of a longitudinal length of the third portion 156c to a longitudinal length of the exposed portion 152 is in a range of about 0.15 to about 0.25. In other embodiments, a ratio of a longitudinal length of the second portion 156b to a longitudinal length of the third portion 156c is in a range of about 5 to about 16.

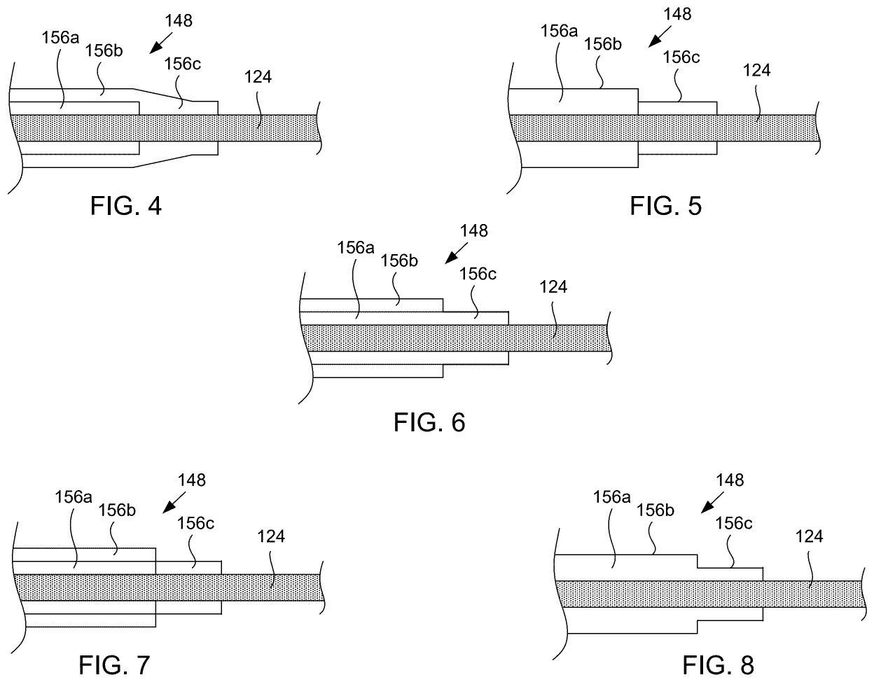

Also, in various embodiments, the first, second, and third portions 156a, 156b, 156c may be separate components from each other, or integral portions of a same component, such as a same tubular member, in any of various combinations. FIGS. 4-8 show example configurations of a cover including three portions, each of which may be representative of an example configuration of the variable thickness cover 148 of FIGS. 1-3.

In a first example configuration shown in FIG. 4, the first portion 156a may be or form a first tubular member, and the second and third portions 156b, 156c may be integral components of a second tubular member separate from the first tubular member. In this context, the second portion 156b is an overlapping portion of the second tubular member in that it overlaps the first tubular member, and the third portion 156c may be a distal overhang of the second tubular member in that it is disposed distal the first tubular member and does not overlap the first tubular member.

In a second example configuration shown in FIG. 5, the first and second portions 156a, 156b may be integral components of a same first tubular member, and the third portion 156c may be or form a second tubular member separate from the first tubular member. In a third example configuration shown in FIG. 6, the first and third portions 156a, 156c may be integral components of a same first tubular member, and the second portion 156b may be or form a second tubular member disposed over the first tubular member. In a fourth example configuration shown in FIG. 7, the first, second, and third portions 156a, 156b, 156c are each separate tubular members from each other. In a fifth example configuration shown in FIG. 8, the first, second, and third portions 156a, 156b, 156c are integral components of a same, single tubular member. In this fifth example configuration, the distal taper may be formed by creating a cutout or otherwise removing a distal portion of the cover 148 to create the multiple outer diameters.

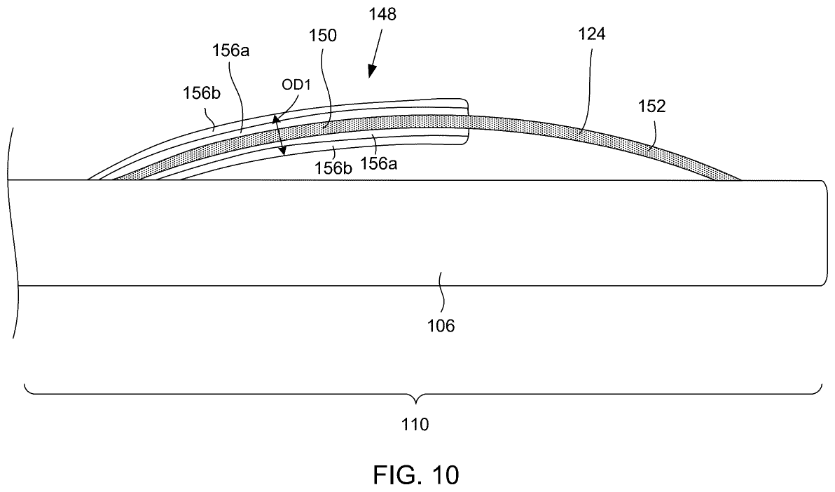

FIG. 10 shows another example configuration of the damage-resistant cover 148. The configuration of the damage-resistant cover 148 in FIG. 10 is a multi-layer cover, in that the first portion 156a comprises a first layer or a first tubular member, and the second portion 156b comprises a second layer or a second tubular member disposed over the first layer/tubular member. In this way, similar to the configurations in FIGS. 3-9, the second portion 156b is an overlapping layer or tubular member that overlaps the first portion 156a, which is directly disposed over the cutting edge 124. Also, similar to the configurations in FIGS. 4, 6, and 7, the first and second portions 156a, 156b may be separate components or tubular members, as opposed to integral components of the same tubular member. For example, the first and second portions 156a, 156b may be affixed or engaged with each other via a mechanical connection, as opposed to being chemically bonded to each other. For example, at least the second portion 156b may be a heat shrink applying an inward bias on the first portion 156b. By being separate layers, as opposed to a single thick tubular cover, damage, such as a crack or other degradation or deterioration, to the second outer layer 156b may not extend to the first inner layer 156a, maintaining the integrity of the first inner layer 156a and the desired protection and insulation provided by the cover 148 overall.

Additionally, similar to the configurations in FIGS. 3-9, in some embodiments, the first and second layers 156a, 156b may be made of the same material. In other embodiments, the first and second layers 156a, 156b may be made of different materials. In addition or alternatively, in any of various embodiments, the first and second layers 156a, 156b may each be made of parylene, polyimide, or polytetrafluoroethylene (PTFE). In addition or alternatively, at least one of the first or second layers 156a, 156b is made of at least one heat shrink material, and/or at least one of the first layer 156a or the second layer 156b is a heat shrink (or heat shrink structure). For example, both the first and second layers 156a, 156b are heat shrinks. As another example, the first layer 156a is a heat shrink but the second layer 156b is not. In another example, the second layer 156b is a heat shrink but the first layer 156a is not.

Additionally, in contrast to the configurations in FIGS. 3-9, the cover 148 in FIG. 10 does not have a third portion 156c disposed distal the first portion 156a. In this way, the second layer 156b does not distally extend past a distal end of the first layer 156a. In some embodiments, the first and second layers 156a, 156b have generally the same longitudinal length, and/or their distal ends are longitudinally aligned or even with each other, such that the distal end of the second layer 156b is disposed over the distal end of the first layer 156a.

The subject matter of the present description may also relate, among others, to the following aspects:
In a first aspect, includes a sphincterotome comprising: an elongate tubular member longitudinally extending from a proximal portion to a distal portion, the elongate tubular member comprising: a body longitudinally extending from the proximal portion to the distal portion; and a cutting wire lumen longitudinally extending in the body; a cutting wire longitudinally extending and movably disposed in the cutting wire lumen, the cutting wire comprising a cutting edge disposed outside of the elongate tubular member, the cutting edge comprising an exposed portion and an insulated portion; a variable thickness cover that covers the insulated portion, the variable thickness cover comprising a distal taper distally extending from a first outer diameter to a second outer diameter, the first outer diameter larger than the second outer diameter.
A second aspect includes the first aspect, and further includes wherein the distal taper comprises a staircase configuration.
A third aspect includes the first aspect, and further includes wherein the distal taper comprises a continuously decreasing outer diameter from the first outer diameter to the second outer diameter.
A fourth aspect includes any of the first through third aspects, and further includes wherein the variable thickness cover comprises: a first portion comprising a first material, the first portion disposed directly over the insulated portion of the cutting edge; a second portion comprising a second material, the second portion disposed over the first portion; and a third portion comprising a third material, the third portion disposed directly over the insulated portion of the cutting edge and disposed distal the first portion, wherein the first portion and the second portion define the first outer diameter, and the third portion defines the second outer diameter.
A fifth aspect includes the fourth aspect, and further includes wherein the first material and the second material are the same.
A sixth aspect includes the fourth aspect, and further includes wherein the first material and the second material are different.
A seventh aspect includes any of the fourth through sixth aspects, and further includes wherein the third material is the same as at least one of the first material or the second material.
An eighth aspect includes the seventh aspect, and further includes wherein the third material is the same as the second material.
A ninth aspect includes any of the fourth through sixth aspects, and further includes wherein the third material is different than both the first material and the second material.
A tenth aspect includes any of the fourth through ninth aspects, and further includes wherein the first material comprises: parylene, polyimide, or polytetrafluoroethylene (PTFE).
An eleventh aspect includes any of the fourth through tenth aspects, and further includes wherein the second material comprises: parylene, polyimide, or polytetrafluoroethylene (PTFE).
A twelfth aspect includes any of the fourth through eleventh aspects, and further includes wherein the third material comprises parylene, polyimide, or polytetrafluoroethylene (PTFE).
A thirteenth aspect includes any of the fourth through seventh aspects, and further includes wherein at least one of the first portion, the second portion, or the third portion comprises at least one heat shrink.
A fourteenth aspect includes the thirteenth aspect, and further includes wherein the first portion and the second portion each comprise the at least one heat shrink.
A fifteenth aspect includes the thirteenth aspect, and further includes wherein the second portion and the third portion each comprise the at least one heat shrink.
A sixteenth aspect includes any of the fourth through fifteenth aspects, and further includes wherein the first portion comprises a first tubular member, and the second portion and the third portion are integral components of a second tubular member that is separate from the first tubular member.
A seventeenth aspect includes any of the fourth through fifteenth aspects, and further includes wherein the first portion and the second portion are integral components of a first tubular member, and the third portion comprises a second tubular member that is separate from the first tubular member.
An eighteenth aspect includes any of the fourth through fifteenth aspects, and further includes wherein the first portion and the third portion are integral components of a first tubular member, and the second portion comprises a second tubular member that is separate from the first tubular member.
A nineteenth aspect includes any of the fourth through fifteenth aspects, and further includes wherein the first portion, the second portion, and the third portion are integral components of a same tubular member.
A twentieth aspect includes any of the fourth through fifteenth aspects, and further includes wherein the first portion, the second portion, and the third portion comprise separate tubular members from each other.
A twenty-first aspect includes any of the fourth through twentieth aspects, and further includes wherein the third portion comprises a longitudinal length in a range of 0.5 millimeters (mm) to 15 mm.
A twenty-second aspect includes any of the fourth through twentieth aspects, and further includes wherein the third portion comprises a longitudinal length in a range of 0.5 millimeters (mm) to 2.5 mm.
A twenty-third aspect includes any of the fourth through twentieth aspects, and further includes wherein the third portion comprises a longitudinal length in a range of 2 mm to 10 mm.
A twenty-fourth aspect includes any of the fourth through twenty-third aspects, and further includes wherein a ratio of a longitudinal length of the third portion to a longitudinal length of the exposed portion is in a range of 0.15 to 0.25.
A twenty-fifth aspect includes any of the fourth through twenty-third aspects, and further includes wherein a ratio of a longitudinal length of the second portion to a longitudinal length of the third portion is in a range of 5 to 16.
A twenty-sixth aspect includes any of the first through twenty-fifth aspects, and further includes wherein the sphincterotome comprises a bipolar sphincterotome.
A twenty-seventh aspect includes any of the first through twenty-fifth aspects, and further includes wherein the sphincterotome comprises a monopolar sphincterotome.
A twenty-eighth aspect includes a sphincterotome comprising: an elongate tubular member longitudinally extending from a proximal portion to a distal portion, the elongate tubular member comprising: a body longitudinally extending from the proximal portion to the distal portion; and a cutting wire lumen longitudinally extending in the body; a cutting wire longitudinally extending and movably disposed in the cutting wire lumen, the cutting wire comprising a cutting edge disposed outside of the elongate tubular member, the cutting edge comprising an exposed portion and an insulated portion; a multi-layer cover that covers the insulated portion, the multi-layer cover comprising a first layer disposed over the insulated portion, and a second layer disposed over the first layer.
A twenty-ninth aspect includes the twenty-eighth aspect, and further includes wherein the first layer and the second layer are made of different materials from each other.
A thirtieth aspect includes the twenty-eighth aspect, and further includes wherein the first layer and the second layer are made of a same material as each other.
A thirty-first aspect includes any of the twenty-eighth through thirtieth aspects, and further includes wherein the first layer is made of: parylene, polyimide, or polytetrafluoroethylene (PTFE).
A thirty-second aspect includes any of the twenty-eighth through thirty-first aspects, and further includes wherein the second layer is made of: parylene, polyimide, or polytetrafluoroethylene (PTFE).
A thirty-third aspect includes any of the twenty-eighth through thirty-second aspects, and further includes wherein at least one of the first layer or the second layer is made of at least one heat shrink material.
A thirty-fourth aspect includes the thirty-first aspect, and further includes wherein the second layer is made of the at least one heat shrink material.
A thirty-fifth aspect includes any of the twenty-eighth through thirty-second aspects, and further includes wherein the second layer comprises a distal overhang that distally extends past a distal end of the first layer and is directly disposed over the insulated portion of the cutting edge.
A thirty-sixth aspect includes the thirty-fifth aspect, and further includes wherein the distal overhang comprises a longitudinal length in a range of 0.5 millimeters (mm) to 15 mm.
A thirty-seventh aspect includes the thirty-fifth aspect, and further includes wherein the distal overhang comprises a longitudinal length in a range of 0.5 millimeters (mm) to 2.5 mm.
A thirty-eight aspect includes the thirty-fifth aspect, and further includes wherein the distal overhang comprises a longitudinal length in a range of 2 mm to 10 mm.
A thirty-ninth aspect includes the thirty-fifth aspect, and further includes wherein a ratio of a longitudinal length of the distal overhang to a longitudinal length of the exposed portion is in a range of 0.15 to 0.25.
A fortieth aspect includes any of the twenty-eighth through thirty-fourth aspects, and further includes wherein the second layer does not distally extend past a distal end of the first layer.
A forty-first aspect includes the fortieth aspect, and further includes wherein a distal end of the second layer is disposed over the distal end of the first layer.
A forty-second aspect includes any of the twenty-eighth through forty-first aspects, and further includes wherein the sphincterotome comprises a bipolar sphincterotome.
A forty-third aspect includes any of the twenty-eight through forty-first aspects, and further includes wherein the sphincterotome comprises a monopolar sphincterotome.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A sphincterotome comprising:
an elongate tubular member longitudinally extending from a proximal portion to a distal portion, the elongate tubular member comprising:
a body longitudinally extending from the proximal portion to the distal portion; and
a cutting wire lumen longitudinally extending in the body;
a cutting wire longitudinally extending and movably disposed in the cutting wire lumen, the cutting wire comprising a cutting edge disposed outside of the elongate tubular member, the cutting edge comprising an exposed portion and an insulated portion;
a variable thickness cover that covers the insulated portion, the variable thickness cover comprising a distal taper distally extending from a first outer diameter to a second outer diameter, the first outer diameter larger than the second outer diameter.

2. The sphincterotome of claim 1, wherein the distal taper comprises a staircase configuration.

3. The sphincterotome of claim 1, wherein the distal taper comprises a continuously decreasing outer diameter from the first outer diameter to the second outer diameter.

4. The sphincterotome of claim 1, wherein the variable thickness cover comprises:
a first portion comprising a first material, the first portion disposed directly over the insulated portion of the cutting edge;
a second portion comprising a second material, the second portion disposed over the first portion; and
a third portion comprising a third material, the third portion disposed directly over the insulated portion of the cutting edge and disposed distal the first portion,
wherein the first portion and the second portion define the first outer diameter, and the third portion defines the second outer diameter.

5. The sphincterotome of claim 4, wherein at least one of the first portion, the second portion, or the third portion comprises at least one heat shrink.

6. The sphincterotome of claim 5, wherein the first portion and the second portion each comprise the at least one heat shrink.

7. The sphincterotome of claim 5, wherein the second portion and the third portion each comprise the at least one heat shrink.

8. The sphincterotome of claim 4, wherein the first portion comprises a first tubular member, and the second portion and the third portion are integral components of a second tubular member that is separate from the first tubular member.

9. The sphincterotome of claim 1, wherein the sphincterotome comprises a bipolar sphincterotome.

10. The sphincterotome of claim 1, wherein the sphincterotome comprises a monopolar sphincterotome.

11. A sphincterotome comprising:
an elongate tubular member longitudinally extending from a proximal portion to a distal portion, the elongate tubular member comprising:
a body longitudinally extending from the proximal portion to the distal portion; and
a cutting wire lumen longitudinally extending in the body;
a cutting wire longitudinally extending and movably disposed in the cutting wire lumen, the cutting wire comprising a cutting edge disposed outside of the elongate tubular member, the cutting edge comprising an exposed portion and an insulated portion;
a multi-layer cover that covers the insulated portion, the multi-layer cover comprising a first layer disposed over the insulated portion, and a second layer disposed over the first layer.

12. The sphincterotome of claim 11, wherein the first layer and the second layer are made of different materials from each other.

13. The sphincterotome of claim 11, wherein the first layer and the second layer are made of a same material as each other.

14. The sphincterotome of claim 11, wherein at least one of the first layer or the second layer is made of at least one heat shrink material.

15. The sphincterotome of claim 11, wherein the second layer is made of the at least one heat shrink material.

16. The sphincterotome of claim 11, wherein the second layer comprises a distal overhang that distally extends past a distal end of the first layer and is directly disposed over the insulated portion of the cutting edge.

17. The sphincterotome of claim 11, wherein the second layer does not distally extend past a distal end of the first layer.

18. The sphincterotome of claim 17, wherein a distal end of the second layer is disposed over the distal end of the first layer.

19. The sphincterotome of claim 11, wherein the sphincterotome comprises a bipolar sphincterotome.

20. The sphincterotome of claim 11, wherein the sphincterotome comprises a monopolar sphincterotome.
